(19) United States
(12) Patent Application Publication
(10) Pub. No.: US 2011/0061664 A1
(43) Pub. Date: Mar. 17, 2011

(54) STOCKING FOR FEET WITH BUNIONS

(76) Inventor: Alberto Paris Mayans Carlos, Bagota (CO)

(21) Appl. No.: 12/991,281

(22) PCT Filed: May 8, 2008
(86) PCT No.: PCT/IB08/01364

§ 371 Date: Nov. 5, 2010

(51) Int. Cl. A61F 13/06; A43B 17/00

(52) U.S. Cl. 128/894; 2/239

(57) ABSTRACT

Stockings for feet with Bunions, the stocking including two sections, the first section being a cavity in the form of a rectangular parallelepiped shape for inserting the big toe or hallux, and the second section being a truncated pyramid-shaped cavity for inserting the other toes; in addition, the stocking has a reinforcement in the first section or cavity of the stocking, surrounding the hallux from the mid section to the lateral section, and from the top to bottom of the hallux, made of the same material as the stocking or of a different material, having a greater fiber density, less elasticity and greater tension than the material of the stocking.

### TECHNICAL ASPECTS

The present application for a Patent of Invention of a Stocking for Bunions, which is designed to separate the big toe of the foot (hallux) from the other toes, keeping it correctly aligned to prevent the sub-dislocation of the metatarsus phalange joint of the hallux, given that it has special reinforcements that prevent this toe from deviating towards the other toes (valgus deviation).

### STATE OF THE ART

Currently there are no descriptions of socks or stockings for the prevention and treatment of the deformity that characterizes bunions, being the latter a pathology of high incidence throughout the world, more predominant in females, with important consequences at the functional level, due to pain, alteration in walking and difficulty in the use of shoes, and at the aesthetic level, due to joint deformity.

The following documents were found regarding patents of invention, in a search conducted in databases and patent compilations that refer to different types of socks or stockings for therapeutic use, like the treatment of varicose veins, lower limb edema, cramps and abnormal fatigue:

Colombian Patent of Invention Application CO 92-109429 titled: “POLYAMIDE FILAMENTS WITH A GREAT CAPACITY FOR RIPPLING ESPECIALLY SUITED FOR THE PRODUCTION OF STOCKINGS” filed on Sep. 23, 1970, and owned by the company E I DU PONT DE NEMOURS AND COMPANY, inventors Anthony Antón and Emil John Volcheck.

Colombian Patent of Invention Application CO 98-26706 titled: “STOCKINGS FOR THERAPEUTIC SUPPORT IN ULCEROVARICOSE PATHOLOGIES” filed on May 13, 1998, by JOSÉ ALVARO PONGUTA GARZÓN, inventor Jorge Ulloa Dominguez. This invention refers to a stocking manufactured from an elastic material with a pre-established elongation, designed to cover from the tarsus to the lower part of the knee, it has a zipper that is slightly displaced to one of the sides with respect to the frontal area, and has several pull-tabs and on the side opposite to the zipper there is an internal pocket; it can be easily adjusted and placed. It is used in the therapeutic support of ulcerovaricose pathologies and it consists of a body (1) that has a shape similar to the normal shape of the lower part of the lower limb, the upper part of said body (1) has a reinforcement (2) of a more dense fabric, and stemming from such reinforcement (2) there is a closure (3) consisting of a zipper that extends to the area close to the ankle; such closure is slightly displaced from the frontal midline to one side, several pull-tabs (4) are located at the longitudinal sides of the zipper. Also, on the lower and internal part of the body (1), there is a pocket (6), and completely joined to the body there is a reinforcement heel (5), which in turn adapts to the foot (8), without a toe-cap, which ends in a reinforcement (7). Present status of this application: declared abandoned by Resolution No. 36794 issued on Oct. 31, 2001.

Colombian Patent of Invention Application CO 94-41028 titled: “IMPROVED STOCKING AND SUPPORT FOR WOMEN” filed by JONES ALMA MARGARET. This invention refers to a set of stockings and supports for women, consisting of: a pair of full-length stockings made of an elastic material and each stocking has fastening elements located only at one place throughout the external part, close to the edge of the upper opening, to be joined by another fastener; and a band that goes around the users torso, this band having only two fasteners, one on the area of each hip, therefore, the fastener of the band can be joined to the external fastener of the stocking, allowing these stockings to be stretched to their complete length, from the foot to the crotch of the user and to the external area of the hip, using a normal mid cut-off point.

U.S. Pat. No. 4,180,065 titled: “ANTI-EMBOLISM STOCKING” granted on Dec. 25, 1979 to BEAR BRAND HOSIERY Co.

U.S. Pat. No. 4,745,917 titled: “THERAPEUTIC STOCKING” granted on May 24, 1988 to THE KENDALL COMPANY.

U.S. Pat. No. 5,412,957 titled: “KNIT THERAPEUTIC STOCKING WITH ANTI-SLIP FEATURE” granted on May 9, 1995 to THE KENDALL COMPANY.

International Patent Application PCT/DK00/00517 titled “SOCK OR STOCKING FOR THERAPEUTIC USE” filed on Sep. 19, 2000 by MERRILD, Bente, Kathe, Yonne and NIELSEN, Ludvig, Nikotaj, Denmark, which was published in the Official Gazette of the PCT under the number WO 01/21119 A1 on Mar. 29, 2001.

U.S. Pat. No. 6,216,495 titled: “SUPPORT STOCKING” granted on Apr. 17, 2001 to BV SPORT.

International Patent Application PCT/US01/49085 titled “THERAPEUTIC STOCKINGS” filed on Dec. 18, 2001 by BEIERS-DORF-JOBST, INC., of the United States, and published in the Official Gazette of the PCT under the number WO 02/49558 A1 on Jun. 27, 2002.

U.S. Pat. No. 6,430,970 titled: “COMPRESSIVE ORTHOSIS SUCH AS RETENTION STOCKING OR TIGHTS” granted on Aug. 13, 2002 to INNOTHERA TOPIC INTERNATIONAL.

International Patent Application PCT/EP02/10632 titled “COMPRESSION SLEEVE FOR TREATMENT OF THE EXTREMITIES” filed on Sep. 21, 2002 by SARIC, Krunoslav; VOLKMANN, Hans-Peter, of Germany, and published in the Official Gazette of the PCT under the number WO 03/028599 A2 on Apr. 10, 2003.

U.S. Pat. No. 6,613,007 titled: “MULTILAYER COMPRESSION STOCKING SYSTEM AND METHOD” granted on Sep. 2, 2003 to CAROLON COMPANY, equivalent to publication in the Official Gazette of the PCT number WO 01/00118 A1 of the International Application PCT/US00/17213 on Jun. 23, 2000.

### ADVANTAGES OF THE INVENTION

The Stocking for Feet with Bunions that constitutes the object of the present invention has the following advantages with respect to the State of the Art:

The stocking of the invention is characterized by separating the first toe of the foot (hallux) from the other toes, given that it has special reinforcements that surround the hallux from the its medial to lateral and from its superior to inferior areas, avoiding the deviation of the hallux towards the other toes.

The stocking is used in the pre and post-surgical treatment of bunions. Moreover, it may be used preventively in cases of incipient hallux valgus, in patients who are genetically predisposed or for the comfort of the user.

The stocking can be manufactured in several materials, provided it complies with the properties of elasticity, in such as a way that it is not deformed by daily use, and hygiene, allowing the absorption of humidity or perspiration from the feet, eliminating the risk of producing fungus and keeping the feet dry.

The stocking of the invention is designed for women as well as for men, in different models (lengths, colors, materials and textures) and with the possibility of being accompanied by other therapeutic and/or aesthetic properties—anti-varicose, gluteus lift, and abdomen control—. It may also have an interior reinforcement, according to the needs of the user, made of a sheet of gel for care of the post-surgical scar, or to decrease pressure on the bunion.

### LISTING OF ENCLOSED FIGURES

FIG. 1. Superior view of the bones of a foot with a bunion.

FIG. 2. Perspective view of the stocking that constitutes the object of the invention.

FIG. 3. Superior view of the stocking of the invention.

FIG. 4. Inferior view of the stocking of the invention.

FIG. 5. Perspective view of the stocking with sheet of gel.

### DESCRIPTION OF THE INVENTION

Bunions, technically known as hallux valgus, are a set of signs and symptoms, where there is a certain degree of inward deviation of the first metatarsal, an outward deviation of the first toe (hallux), a deformity by rotation of the first toe, the lateral displacement of the sesamoids and tendon contractures (mainly of the adductor of the hallux and of the medial capsule (with respect to the axis of the foot)).

The causes or factors responsible for bunions are diverse:
Inheritance

If there are precedents of bunions in a family, it is very likely that this predisposition will be inherited and hence there will be a high probability of developing them. Juvenile bunions, or that which arises during adolescence, usually has hereditary characteristics. Statistics prove that 63% of individuals with bunions have an immediate relative with bunions. Generally, bunions are caused by a hereditary defect in the mechanical structure of the foot. Bunions themselves are not hereditary; however, certain types of feet are more prone to develop bunions.
Footwear

Footwear is one of the elements responsible for the appearance of bunions. There are studies that compare social groups that are always barefoot to social groups that use shoes. Social groups that are permanently barefoot have a lower incidence of bunions. 33% of individuals that use shoes have bunions, in comparison to 2% of barefoot individuals.

Footwear has two kinds of effects: on one hand, it exerts pressure on the front part of the foot confining it to a reduced space and, on the other hand, the toes move less than if barefoot, and this reduced mobility of the toes gives rise to an atrophy of the muscles of the foot. This decrease of strength in the muscles causes an imbalance in the forces that govern the direction of the toes.

Due to the above, bunions are very common for women between the ages of 40 and 60 who wear or have worn high heel shoes or shoes with a narrow tip. The fashion of narrow shoes that began after World War II caused an increase in this pathology. The foot is placed on an inclined plane, on which it tends to be displaced forward. The front part of the foot experiences an overload; the toes hit against the shoe and shrink against the front tip. The posture of the skeleton of the foot is extended and laterally compressed, forcing the toes to cram. The whole foot is in a mold that deforms its position and molds it in this bad posture through compression.

Conventional Treatments for Feet with Bunions

When symptoms are not extreme, the patient may be concerned by the aesthetic aspects. The treatment of bunions must address the symptomatic aspects as well as the aesthetic aspects. Considering the general lengthy nature of this pathology and its possible hereditary and degenerative etiology, a conservative therapy should be initiated. Special footwear may be prescribed for bunions to make room for the deformity.

Pathologies of the foot that are candidates for surgery as their only form of treatment reach 40% in a population with an average age of 65. In a study carried out in a hospital in Valencia, it was found that between the infantile and juvenile population (4-17 years of age) 55.5% was affected by foot pathologies, although these, at such age, are generally treated without surgery and correctively or preventively. Whereas, if for example we refer to the elderly, pathologies of the foot with a surgical solution make up 52% of the cases.

Bunions affect specifically 6-10% of the general population and it is the most frequent pathology of the foot, particularly among women (20% of adult women and 34% of elderly women).

Avoiding surgery is the first objective when seeking the relief of a person with bunions.

To relieve the pain caused by bunions, there are several treatments and recommendations, which will not reverse the deformity of the toe, such as the following:
a) Change of footwear. It is very important to use the adequate type of footwear. Shoes that are broad fitting in the area of the toes should be selected and pointed or high-heel shoes should be avoided, given that these can worsen the pathology. Footwear should be modified, it is convenient that they do not constrict the feet and that they have a soft texture and a heel that does not exceed 5 cm.
b) Pads. These are placed on the area of the bunion helping to minimize the pain. It is important to try to relieve pain, inflammation of the bursa and calluses, eliminating friction on the lateral prominence. Day and night separators should be used.
c) Activities that cause pain on the bunions should be avoided, including standing for long periods.
d) Non-steroid anti-inflammatory medications help relieve the pain.
e) Applying bags of ice several times a day helps decrease inflammation and pain.
f) Injection therapy. Injections of corticoid-steroids may aid in the treatment of inflammation of the bursa (fluid sac located in joints) that sometimes appears with bunions, although these injections are rarely used in the treatment of bunions.
g) Orthopedic devices. In some cases, the orthopedic trauma specialist can recommend common orthopedic devices to align the hallux, but they are usually used at night.
h) If the bunion is accompanied by a flat foot, the latter should be improved, usually by the use of an insole.
i) In cases where the patient has a retraction of the Achilles tendon, it is advisable to practice stretching this tendon.

### Post-Surgical Treatment

The patient with surgery is able to walk right after the surgery. Such patients usually do not have any pain, and if pain occurs, it is minimal. Therefore, depending on the patient's profession, he/she may begin working right away or progressively. However, given that the surgery is corrective and such correction should strengthen, shoes that are wide and open at the front must be used. This circumstance may prevent patients from returning to their activities because of their need to be well dressed for their jobs.

The incisions of the bone heal progressively. After 3 weeks, the unions are fibrous, after 6 weeks they are osseous. This means that after 3 weeks the fibrous callous is solid enough to allow more movement. At four weeks, the patient may begin to use wide footwear, such as sports shoes. After 6 weeks, the patient may begin to use regular footwear.

### DETAILED DESCRIPTION OF THE INVENTION

In accordance with the above, several experiments were carried out, with the purpose of finding a new solution, arriving at the creation of the Stocking for Feet with Bunions, which is the object of the present application for a Patent of Invention, consisting of a stocking (1) formed by two sections, the first section (2) is a cavity in the form of a rectangular parallelepiped shape for inserting the big toe or hallux, and the second section (3) is a truncated pyramid-shaped cavity for inserting the other toes.

Furthermore, said stocking has a reinforcement (4) on the first section or cavity (2) of the stocking, surrounding the hallux from the mid section to the lateral section, and from the top to bottom of the hallux; made of the same material as the stocking or from a different material, having a greater fiber density, less elasticity and greater tension than the material of the stocking.

The reinforcement (4) may optionally have an additional internal reinforcement (5), consisting of a soft sheet of silicone gel, for care of the post-surgical scar or for decreasing pressure on the bunion.

The first section (2) allows separation of the first toe or hallux from the other toes or second section (3).

The reinforcement (4) allows improving the alignment of the first toe and avoids pressure zones on the painful sites.

The material of the stocking is a mix of several materials, selected from a group consisting of cotton, rayon, polyester, nylon, acrylic, acrylic micro-fibers and combinations of these mixes of synthetic fibers with great elasticity, such as elastomeric fibers; polyurethane fibers, like elastene or a long-chain synthetic polymer of a segmented polyurethane (spandex); polyamides, like nylon; copolymers of polyacrylonitrile; provided the material complies with the following technical requirements:
Being hygienic, allowing the absorption of humidity or sweat from the feet, and eliminating the risk of producing fungus, keeping the feet dry.
Being durable and comfortable during its use.
Easy to wash, that dirt does not adhere strongly to its synthetic fiber, therefore the latter should be smooth.

The reinforcement (4) of the stocking is manufactured in the same material of the stocking or from a different material, which can be selected from a group consisting of cotton, rayon, polyester, nylon, acrylic, acrylic micro-fibers and combinations of these mixes of synthetic fibers, such as elastomeric fibers; polyurethane fibers, like elastene or a long-chain synthetic polymer of a segmented polyurethane; polyamides; copolymers of polyacrylonitrile.

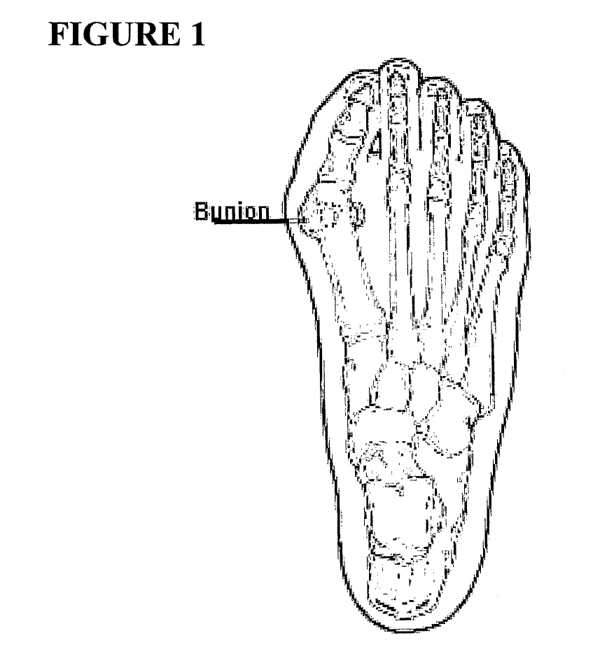

FIG. 1 shows a superior view of the bones of a foot with a bunion (Hallux Valgus) and the area of the bunion.

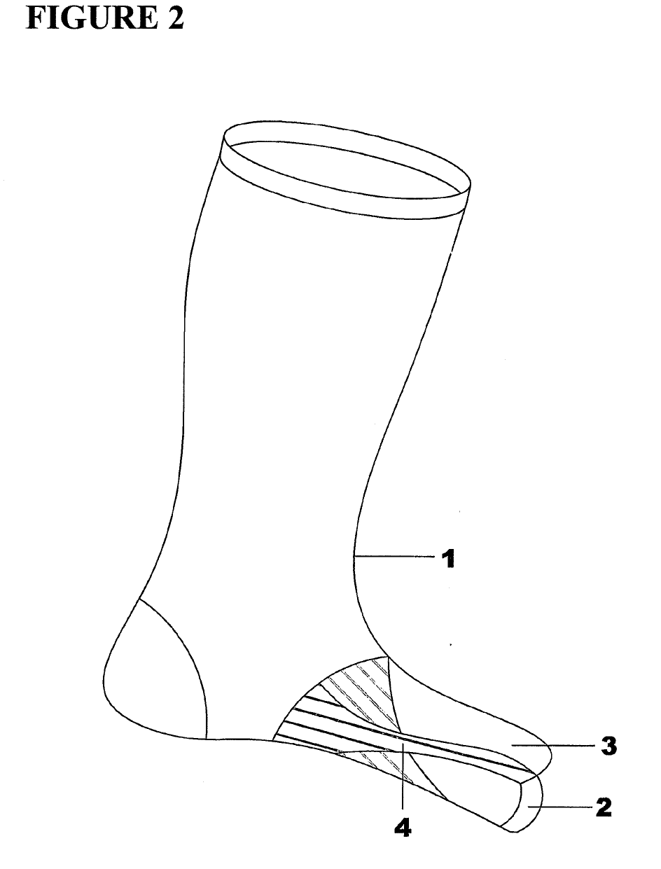

FIG. 2 shows a perspective view of the stocking that constitutes the object of the invention, showing the stocking (1); the first section or cavity (2); the second section (3) and the reinforcement (4).

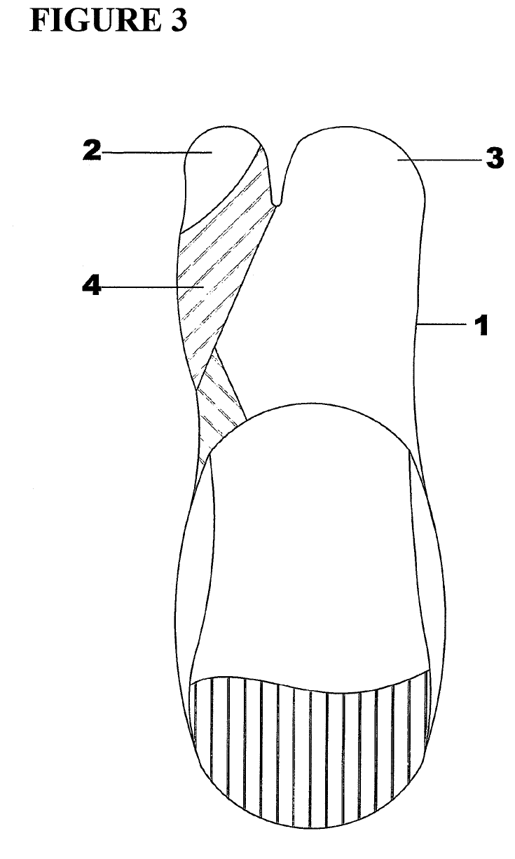

FIG. 3 shows a superior view of the stocking of the invention, showing the stocking (1), the first section or cavity (2), the second section (3) and the reinforcement (4).

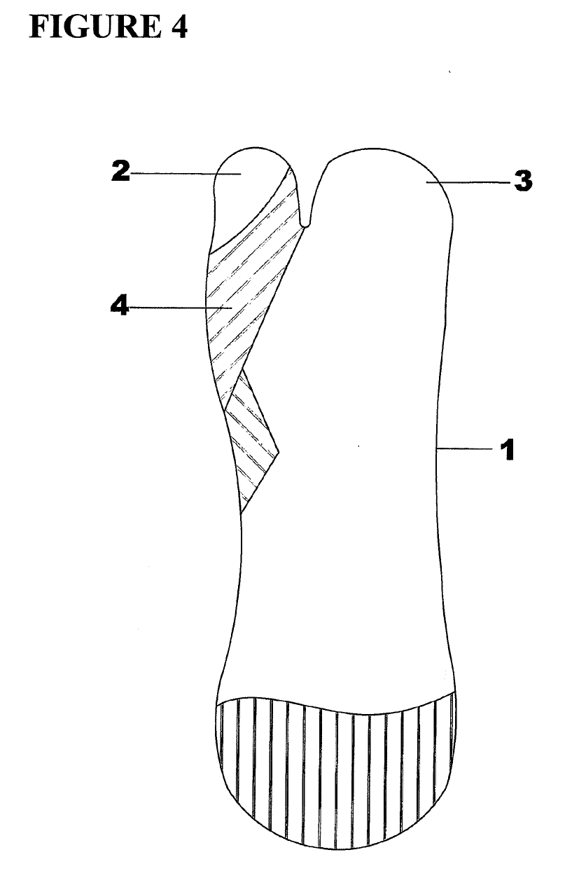

FIG. 4 shows an inferior view of the stocking of the invention, showing the stocking (1) with the first section or cavity (2), the second section (3) and the reinforcement (4).

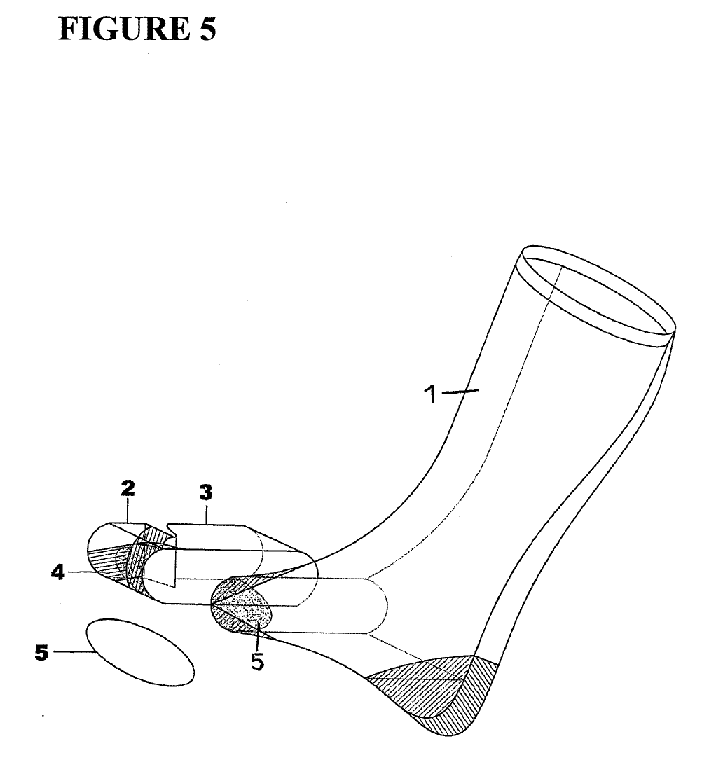

FIG. 5 shows a perspective view of the stocking of the invention, showing the stocking (1) with the first section or cavity (2), the second section (3), the reinforcement (4) and the interior reinforcement (5) consisting of a sheet of silicone gel.

The Stocking is used for the pre and post-surgical treatment of bunions. Moreover, it may be used preventively in cases of incipient hallus valgus, in patients that are genetically predisposed to hallux valgus or for the comfort of the user.

The Stocking can be manufactures in several materials, provided it complies with the properties of elasticity, not being deformed by daily use; and hygiene, allowing the absorption of humidity and sweat from the feet, eliminating the risk of producing fungus and keeping the feet dry.

The Stocking of the invention is designed for men and women, in different models (lengths, colors, materials and textures) and with the possibility of being accompanied by other therapeutic or aesthetic properties—anti-varicose, gluteus lift, and abdomen control—. It may also optionally have an interior reinforcement, according to the needs of the user, made of a sheet of gel for the post-surgical care of the scar, or to decrease pressure on the bunion.

1. A stocking for feet with bunions, comprising: a stocking having two sections, the first section is a cavity in the form of a rectangular parallelepiped shape for inserting the big toe or hallux, and the second section is a truncated pyramid-shaped cavity for inserting the other toes; in addition, the stocking has a reinforcement in the first section or cavity of the stocking, surrounding the hallux from the mid section to the lateral section, and from the top to bottom of the hallux, made of the same material as the stocking or of a different material, having a greater fiber density, less elasticity and greater tension than the material of the stocking.

2. The stocking of claim 1, further including the stocking optionally having, in addition to the reinforcement, an interior reinforcement made of a sheet of silicone gel placed at the level of the bunion.

3. The stocking of claim 1, wherein the stocking is manufactured from several materials, selected from a group consisting of cotton, rayon, polyester, nylon, acrylic, acrylic micro-fibers and combinations of these mixes of synthetic fibers, like elastomeric fibers; polyurethane fibers, like elastene or a long-chain synthetic polymer of a segmented polyurethane; polyamides; copolymers of polyacrylonitrile.

4. The stocking of claim 1, wherein the reinforcement of the stocking is manufactured from the same material of the stocking or from a different material, which is selected from a group consisting of cotton, rayon, polyester, nylon, acrylic, acrylic micro-fibers and combinations of these mixes of synthetic fibers, like elastomeric fibers; polyurethane fibers, like elastene or a long-chain synthetic polymer of a segmented polyurethane; polyamides; copolymers of polyacrylonitrile.

5. The stocking of claim 3, wherein the materials of the stocking must comply with the properties of elasticity and humidity absorption.

6. The stocking of claim 1, wherein the stocking is configured for being used for the pre and post surgical treatment of bunions.

7. The stocking of claim 1, wherein the stocking is configured for being used for the prevention of incipient hallus valgus.

8. The stocking of claim 1, wherein the stocking is configured for being used in patients who are genetically predisposed to hallus valgus.

9. The stocking of claim 1, wherein the stocking is configured for being used in combination with other therapeutic and/or aesthetic properties, such as anti-varicose, gluteus lift and abdomen control properties.

10. The stocking of claim 2, wherein the interior reinforcement, which includes a soft sheet of silicone gel, is configured for being used for care of the post-surgical scar or to decrease pressure on the bunion.
